(19) United States
(12) Patent Application Publication
(10) Pub. No.: US 2006/0094979 A1
(43) Pub. Date: May 4, 2006

(54) Body impedance measurement apparatus

(75) Inventors: Yoshihisa Masuo, Kyoto-shi (JP); Kazuhiko Yoshida, Kyoto-shi (JP)

Correspondence Address: OLIFF & BERRIDGE, PLC, P.O. BOX 19928, ALEXANDRIA, VA, 22320, US

(73) Assignee: Art Haven 9 Co., Ltd., Kyoto-shi (JP)

(21) Appl. No.: 11/311,337

(22) Filed: Dec. 20, 2005

Related U.S. Application Data
(62) Division of application No. 10/450,051, filed on Jun. 10, 2003.

(30) Foreign Application Priority Data
Dec. 14, 2000 (JP) 2000-379977

(51) Int. Cl. A61B 5/05

(52) U.S. Cl. 600/547

(57) ABSTRACT

A relay 201 is provided for opening and closing the signal path connecting the measurement circuits and an electrode, and a relay 213 is provided for opening and closing a DC power line extending from an AC-DC adaptor 3 connected to a commercial AC power supply 5. In the measurement of the impedance, before supplying the current through the body of the subject, the relay 213 is opened to disconnect the commercial AC power supply 5 from a main unit 2 and a personal computer 1 so that the circuits are driven by the DC power from a battery 102. After that, the relay 201 is closed to connect the measuring circuit with the electrodes 10, 11, a weak current is passed through the body of the subject via the electrodes 10, and a voltage generated in the body by the current is measured with the electrodes 11. After the measurement, the relay 201 is opened, the relay 213 is closed, and body composition information is calculated and displayed by performing a calculation based on the measurement value of the impedance and body specific information. Thus, the muscle mass or other information of the subject can be accurately obtained, while electric shock is assuredly prevented during the measurement.

This is a Division of application Ser. No. 10/450,051 filed Jun. 10, 2003, which in turn is a National Stage of PCT/JP01/10806, filed Dec. 10, 2001. The entire disclosure of the prior applications is hereby incorporated by reference herein in its entirety.

### TECHNICAL FIELD

The present invention relates to a body impedance measurement apparatus for measuring bioelectrical impedance of the body of a subject to estimate and present various kinds of information about the body composition (such as the body-fat mass, muscle mass, bone mass, bone density, lean body mass, body-fat ratio and basal metabolic rate) and/or health condition of the subject, using not only the measurement value of the impedance but also the height, weight, age, gender and other information, which will be generally referred to as “body specific information” hereinafter.

### BACKGROUND ART

Conventionally, the most common method in health management concerning obesity or other body conditions is to measure body weight. Nowadays, obesity is not regarded simply as a body type, but people are looking at indices for measuring obesity. One such index is the body-fat mass, which indicates the mass of subcutaneous fat and/or visceral fat; another is the body-fat ratio, which indicates the ratio of body-fat to body weight.

Many parties have been studying methods of measuring the bioelectrical impedance of the body (simply referred to as the “impedance” hereinafter) and estimating body-fat ratio and/or other values based on the impedance. One such method is a four-electrode method, which uses a pair of current-carrying electrodes attached to the back of the right hand and the instep of the right foot of the subject, respectively, and a pair of measuring electrodes attached to some parts between the current-carrying electrodes, such as the right wrist and the right ankle. With a radio-frequency current being supplied through the body between the current-carrying electrodes, the potential difference between the measuring electrodes is measured. Then, the impedance is calculated from the voltage value and the current value, and the body-fat ratio and/or other information are estimated based on the measurement value of the impedance.

Recently, more convenient apparatuses for measuring body-fat ratio (so-called “body-fat meters”) have been developed, and are commercially available. An example of such apparatuses is disclosed in the Japanese Unexamined Patent Publication No. H7-51242. This apparatus includes a pair of grips to be held with both hands, each grip provided with a current-carrying electrode and a measuring electrode. The electrodes are located so that, when the subject holds the grips with both hands, the current-carrying electrode contacts the finger-side part of the hand and the measuring electrode contacts the wrist-side part of the hand. Then, based on the bioelectrical impedance measured thereby, the apparatus estimates various kinds of information, such as lean body mass, body-fat ratio, total body water or basal metabolic rate. Another example is disclosed in the Japanese Examined Patent Publication No. H5-49050. This apparatus has a measuring platform, on which the above electrodes are located to contact the soles of the feet when the subject steps on it, so that the weight and the body-fat can be measured simultaneously.

The above-described apparatuses measure the impedance with the current path extending between one hand and one foot, between both hands or between both legs. In the case of measuring the voltage with the current path between one hand and one foot, the current path includes the chest or abdomen (i.e. trunk), whose cross-sectional area is much larger than that of the leg or the arm. In this case, the contribution of the leg or the arm to the impedance is relatively great while that of the subcutaneous fat of the abdomen or the intra-abdominal fat (visceral fat) is relatively small. This means that the measurement result hardly reflects the increase or decrease in the subcutaneous fat of the abdomen or the intra-abdominal fat, so that the result lacks reliability. In the case of measuring the voltage with the current path between both hands or both feet, on the other hand, most of the trunk is excluded from the current path, so that the error in the estimation of the body-fat ratio or other indices for the entire body is likely to be large.

Conventionally, when the body-fat ratio or other indices are estimated from the measurement value of the impedance, an estimation formula for the bioelectrical impedance approach (BIA), created according to a calibration curve prepared using the underwater weighing method as the estimation basis, is used. By this method, however, it is impossible to decrease the estimation error because the method has some faults, such as lack of consideration of the difference in the contribution of the muscle, bone or other lean body components to the impedance.

The calculation of body composition from the impedance is based on the assumption that the human body can be modeled as a structure composed of three kinds of tissue: bone, muscle and fat, which have different electrical characteristics. In this model, it is assumed that the three tissues are connected in parallel, the component ratios of the tissues are fixed, and the whole component tissues and each tissue have fixed electrical characteristics (i.e. volume resistivity). Various statistical researches suggest that this assumption is highly reliable for normal adults. However, for children, minors, elderly people, athletes or other groups of people having special body composition, it is impractical to obtain reliable results because there are personal differences in the component ratios and electrical characteristics, which often make the aforementioned values deviate from the above condition.

In respect not to the prevention of obesity, but in respect to the determination of the progress of strengthening the body or the progress of aging, it is very important to measure the muscle mass or muscle force of the body. For example, an athlete or similar person intending to improve the ability of the body will not only regard the muscle mass as an index for measuring the effect of training or other activities but also use the index to set an objective for training. This observation applies also for the case of a person in the process of rehabilitation for the strengthening and recovery of a part of the body that has been weakened after a long hospital stay due to an injury or illness. Furthermore, the expected increase of elderly people will make it necessary to easily measure the muscle mass, muscle force and right-and-left side balance of such properties of muscles for each elderly person in nursing care. The information obtained by the measurement will make it possible to evaluate the ability of the person to live an independent life, and to provide an improved living environment and diet plan (meals, exercises) that are designed to supplement any inconvenience or shortage in daily life so that the person can exhibit a high performance in daily activity.

Conventional apparatuses in this field cannot provide aforementioned kinds of information or can merely provide such information with inadequate accuracy.

It is of course possible to perform accurate measurements by using a magnetic resonance imaging apparatus or an X-ray computed tomography scanner, which are generally used in large hospitals. These apparatuses, however, occupy large spaces and are very costly. Furthermore, the apparatuses keep the subject in the bound state and impose physical and mental burdens on the subject, regardless of their age.

Preferably, the apparatus should be small enough for welfare workers or similar persons to take it with them when visiting elderly people, and easy enough to perform measurements on the subjects at their homes. In other words, it is desirable that well-trained persons can perform measurements on the subject with the apparatus without difficulty. It is also desirable that the apparatus does not require extraordinary production costs. Then, the apparatus will be of greater value, even if it is not easy for everybody to use it.

In view of the above problems, the applicant has proposed a new body composition measurement method and apparatus, as described in the Japanese Patent Application No. 2000-362896. The body composition measurement method and apparatus divide the human body into plural parts and estimate body composition information, such as the muscle mass, bone mass and fat mass, of each body part with high accuracy. To improve the accuracy of the measurement with such apparatuses, attentions should be paid to the details of the apparatus. For example, the influence from various external noises coming from the air or transmitted through power lines or other media must be removed to the utmost. Such a requirement is almost ignorable for conventional body composition measurement apparatuses with relatively low accuracy. On the other hand, for apparatuses having high measurement accuracy, such as the one proposed by the applicant, the above requirement is crucial.

In normal measurement, the above body composition measurement apparatus supplies such a weak current through the body that does not damage the body tissues. It is necessary to provide the apparatus with a means for preventing an excessive amount of current from flowing via the electrodes into the body of the subject and damaging the body in the event an abnormality has occurred to the apparatus.

The above body composition measurement apparatus may be used in a place such as medical facilities where an enough space is available for installation. However, it should be considered that some type of the apparatus should be used in a relatively small space. For example, care workers or welfare workers visiting elderly people living alone may use the apparatus on the spot to perform the measurement. Also, the apparatus must be portable to a certain extent.

The present invention has been accomplished in view of the above problems, the first object of which is to propose a body impedance measurement apparatus capable of accurately measuring the body impedance to obtain various kinds of information about body composition, such as body-fat mass and muscle mass, and/or health condition. Another object of the present invention is to propose a high-safety body impedance measurement apparatus capable of assuredly protecting the subject from electric shock or other accidents. Still another object of the present invention is to propose a body impedance measurement apparatus that occupies only a small space and is easy to carry.

### DISCLOSURE OF THE INVENTION

To solve the above problems, the present invention proposes, as the first invention, a body impedance measurement apparatus including a measuring unit for supplying a weak current through the body of a subject from current-carrying electrodes attached to the body and measuring a voltage generated by the current with measuring electrodes attached to the body, and a calculating unit for calculating an impedance of the body from the value of the current supplied through the body and the value of the voltage measured, including:

a) a power converter for converting AC power supplied from a commercial AC power supply to DC power;

b) a storage battery for storing the DC power and supplying the DC power as a driving power for the apparatus at least when the AC power is not supplied;

c) a power line opening/closing means for opening or closing a power line connecting the commercial AC power supply to the power converter or a power line connecting the power converter to the storage battery; and

d) a controller for opening the power line opening/closing means so that the driving power is supplied from the storage battery to each circuit of the apparatus at least during a period of time for measuring the voltage with the current supplied through the body.

In the body impedance measurement apparatus according to the first invention, the controller closes the power line opening/closing means to connect the commercial AC power supply to the apparatus when there is no need to supply the current through the body, e.g. during a preparation period for entering and setting information before the measurement, during a standby period before the measurement, during a period of calculating the impedance after the measurement of the voltage, and/or during a period of estimating the muscle mass, fat mass, bone mass and/or other body composition information of the entire body or a part of the body of the subject, using the measurement value of the impedance calculated and/or the height, weight and/or other body specific information of the subject. There, the power converter converts the AC power supplied from the commercial AC power supply to a DC power having an appropriate voltage. Circuits of the measuring unit and the calculating unit are supplied with the DC power as the driving power.

In response to a measurement start command, the controller opens the power line opening/closing means to disconnect the commercial AC power supply from the apparatus before supplying a current via the current-carrying electrodes through the body so that the DC power from the storage battery is supplied as the driving power for the apparatus. When the measurement of the voltage with the current supplied through the body of the subject is completed and the current supply is accordingly stopped, the controller closes the power line opening/closing means to re-connect the commercial AC power supply to the apparatus so that the driving power is switched from the storage battery to the DC power produced by the power converter. Of course, the apparatus can be driven by the DC power supplied from the storage battery even when the power line opening/closing means is closed, if the power cannot be supplied from the commercial AC power supply for some reason, such as when the apparatus is not plugged in an outlet of the commercial AC power supply or if the power supply is cut off. It should be noted that the “period of time for measuring the voltage with the current supplied through the body” may include not only the period of time when the current is actually supplied through the body but also an appropriate length of time preceding or succeeding the aforementioned period of time.

According to the construction of the first invention, the commercial AC power supply is disconnected from the apparatus while the voltage corresponding to body impedance is measured. This prevents the intrusion of external noises through the power line of the commercial AC power supply and improves the signal to noise ratio of the measurement value. Accordingly, the impedance can be calculated with high accuracy. Even when trouble or a malfunctioning of a circuit has occurred, the commercial alternating current never flows into the body of the subject in the process of supplying a current through the body. Thus, the electric shock of the subject is prevented and a high degree of safety is ensured.

The body impedance measurement apparatus according to the first invention may further include a signal line opening/closing means for opening or closing signal lines each connecting a measurement circuit included in the measuring unit with the current-carrying electrodes and the measuring electrodes, where the controller opens the signal line opening/closing means to disconnect the current-carrying electrodes and the measuring electrodes except for the period of time for measuring the voltage with the current supplied through the body.

According to this construction, the current-carrying electrodes and the measuring electrodes are disconnected from the measuring circuit when no current is supplied to the body. Therefore, even if the circuit should have a problem that might allow the commercial alternating current to reach the current-carrying electrode or measuring electrode despite the absence of the command for supplying current, the commercial alternating current is prevented from flowing through the current-carrying electrode or measuring electrode into the body of the subject. Thus, a higher degree of safety is ensured.

In the above construction, the controller may be preferably constructed to open the power line opening/closing means to disconnect the commercial AC power supply from the apparatus and then close the signal line opening/closing means to connect the current-carrying electrodes and the measuring electrodes to the measuring circuit unit before supplying the current through the body, and to open the signal line opening/closing means to disconnect the current-carrying electrodes and the measuring electrodes from the measuring circuit unit and then close the power line opening/closing means to connect the commercial AC power supply to the apparatus after the current supply is ended. By this construction, the commercial AC power supply is always disconnected from the apparatus while the current-carrying electrodes and the measuring electrodes are connected to the measuring circuit unit. Thus, the highest degree of safety is ensured.

The power line opening/closing means and/or the signal line opening/closing means may be constructed using an electromagnetic relay. This not only ensures the on/off switching of the line but also reduces the production costs.

The power line opening/closing means and/or the signal line opening/closing means may be preferably constructed using an electromagnetic relay that requires no driving current to open or close when the current is supplied through the body. For example, the power line opening/closing means should be opened while the current is supplied through the body. Therefore, it is preferable to use a “normal break” type electromagnetic relay, which requires no driving current to open. The signal line opening/closing means, on the other hand, should be closed while the current is supplied through the body. Therefore, it is preferable to use a “normal make” type electromagnetic relay, which requires no driving current to close. While a current is supplied through the body of the subject, the apparatus is driven by the DC power supplied from the storage battery. The above construction is advantageous in that less power is consumed when the storage battery is used.

In a mode of the body impedance measurement apparatus according to the first invention, a predetermined control program is executed on a multi-purpose personal computer to implement the calculation process of the calculating unit, and the measuring unit is enclosed in a main unit contained in a casing with the personal computer. The main unit and the personal computer are capable of communicating with each other.

The above construction makes it possible to obtain the present apparatus by simply installing a predetermined control program in a personal computer and connecting the personal computer with the main unit. Use of a personal computer, a mass-produced product, reduces the production costs of the apparatus. Users may use their own computers to use the apparatus at still lower costs. The “personal computer” includes any computers regardless of their types, such as a notebook or desktop, and also includes any apparatus having a central processing unit (CPU) and capable of functioning as an information-processing terminal substantially equivalent to a personal computer, in which the control program can be externally installed.

Notebook-size personal computers have a built-in battery. Taking this into account, the apparatus may use a built-in battery of a notebook-size personal computer as the storage battery. This eliminates the necessity of using a special battery.

The apparatus may include a serial interface as a communicating means between the personal computer and the main unit. Nowadays, interfaces compliant with the Universal Serial Bus (USB) standard are widely used for connecting peripheral devices to personal computers. The USB standard defines that each port should be capable of providing the maximum power of 5V/500 mA. Accordingly, the aforementioned communication means may be an interface compliant with the USB standard, through which the main unit receives its driving power from the personal computer. This construction makes it possible to bundle a power cable and a signal cable into one cable between the personal computer and the main unit, whereby the connection is facilitated.

In the case of printing a measurement result or other information computed by the personal computer, if a cable is used to connect a printer (or printing means) to the personal computer, then the ground of the personal computer is connected to that of the printer, which may cause an intrusion of a noise from the power source of the printer (e.g. commercial AC power supply). Therefore, it is preferable to use a wireless communication interface between the personal computer and the printing means. For example, various types of infrared communication interfaces generally used are usable. Interfaces using electromagnetic waves are also available.

To solve the aforementioned problems, the present invention proposes, as the second invention, a body impedance measurement apparatus including a measuring unit for supplying a weak current through the body of a subject from current-carrying electrodes attached to the body and measuring a voltage generated by the current with measuring electrodes attached to the body, and a calculating unit for calculating an impedance of the body from the value of the current supplied through the body and the value of the voltage measured, wherein:

the same number of the current-carrying electrodes and the measuring electrodes are used, each current-carrying electrode is paired with each measuring electrode, the first wire of a cable constructed as a balanced type shield wire is used as a signal line for connecting the current-carrying electrode and a measuring circuit unit, the second wire of the cable is used as a signal line for connecting the measuring electrode and the measuring circuit unit, and a plurality of the cables have the same specifications.

To measure body impedance with the second body impedance measurement apparatus, it is necessary to use at least two current-carrying electrodes and two measuring electrodes. The two (or more) signal lines for connecting the measuring electrodes and the measuring circuit unit are each enclosed in two cables each covered by a separate shield. This construction reduces the capacitance between the two measuring electrodes. Furthermore, the shield prevents the interference of external noises. In the case of wire breaking or other trouble, the cable can be replaced with a new one at low costs because the plural cables have the same specifications.

An expandable sheath material may be used as an insulator for separating the two wires of the balanced type shield wire. This not only reduces the capacitance but also provides the cable with desired flexibility, lightweight properties and durability. A preferable example is expandable polystyrene resin, the expansion ratio of which is preferably 75 to 80%.

The current-carrying electrode and the measuring electrode connected to the first and second wires of the same cable may be attached to the same part of the four limbs of the body of the subject. This makes it possible to shorten the lines taken out from the balanced type shield wire and connected to the current-carrying electrode and the measuring electrodes, whereby the interference of external noises can be minimized.

When the current-carrying electrodes and the measuring electrodes are to be attached to the right hand, left hand, right foot and left foot, it is necessary to use four cables to connect these electrodes to the measuring circuit unit. In this case, the cables may preferably have the same length. This design almost equalizes the capacitances of the cables, and accordingly equalizes the influence from the capacitances when the selection of the current-carrying electrodes and the measuring electrodes is changed to alter the current-supplying points and/or voltage-measuring points.

In whatever manner a cable is constructed to reduce its capacitance, it is practically impossible to make the capacitance completely zero. In a very accurate measurement, even a small capacitance may cause an error.

Accordingly, the present invention proposes, as the third invention, a body impedance measurement apparatus including a measuring unit for supplying a weak current through the body of a subject from current-carrying electrodes attached to the body and measuring a voltage generated by the current with measuring electrodes attached to the body, and a calculating unit for calculating an impedance of the body from the value of the current supplied through the body and the value of the voltage measured, wherein:

the input impedance of the measuring unit observed from the measuring electrode is measured by using a reference resistance and capacitance determined beforehand, a correction formula for canceling the influence of the input impedance is created from the input impedance measured and the reference resistance and capacitance, and the calculating unit corrects the value of the impedance calculated on the basis of the result of a measurement by the measuring unit by using the correction formula.

The input impedance contains the capacitance of a cable connecting the measuring electrode and the measuring unit. The input impedance also contains the input impedance of the measuring circuits of the measuring unit.

The above construction almost entirely removes the influence from the input impedance observed from the measuring electrode, including the capacitance of the cable and the input impedance of the measuring circuits of the measuring unit. Therefore, the impedance of the body of the subject can be accurately measured.

The fourth invention proposes a body impedance measurement apparatus including a measuring unit for supplying a weak current through the body of a subject from current-carrying electrodes attached to the body and measuring a voltage generated by the current with measuring electrodes attached to the body, and a calculating unit for calculating an impedance of the body from the value of the current supplied through the body and the value of the voltage measured, wherein:

a measuring circuit unit of the measuring unit is enclosed in a rectangular body casing, and a predetermined control program is executed on a notebook-size personal computer to implement the calculation process of the calculating unit, and

the body casing has, on its top, a recess capable of containing a peripheral device to be connected to the notebook-size personal computer, and the notebook-size personal computer can be placed on the top of the body casing with the peripheral device contained in the recess.

Taking into account their portability, notebook-size personal computers are designed to have small-sized, thin bodies, where some peripheral devices are often designed as a separate unit to be connected to the computer body via a cable, not as a built-in unit. Examples of such devices are external storage devices, such as a floppy-disk drive or CD-ROM drive. In the apparatus according to the fourth invention, when the peripheral device is dropped in a recess formed on the top of the body casing, the top of the peripheral device comes to a level equal to or lower than the surrounding roof-top area of the body casing. Therefore, it is possible to place the notebook-size personal computer on the top of the body casing and use them in the stacked state.

When the recess is formed on the top of the notebook-size personal computer and the peripheral device is set in the recess, the connector terminal of the peripheral device is preferably oriented to the same direction as that of the personal computer. This configuration allows the connection of the both terminals by a cable while the peripheral device is contained in the recess. In this case, the recess is used not only for a storing space of the peripheral device while not in use, but also as a bay for accommodating the peripheral device while in use, which reduces occupation area of the apparatuses in use.

The recess may have a capacity of simultaneously containing both the peripheral device and a cable for connecting the peripheral device and the notebook-size personal computer, and be provided with a positioning element for positioning the peripheral device and a cable holder to or from which the cable is attachable or detachable. Allowing the connecting cable to be stored in the recess when the peripheral device is not used, the above construction not only eliminates the possibility of missing the cable but also improves the appearance.

The top of the body casing may have an area larger than the bottom of the notebook-size personal computer and be provided with a computer-positioning element for positioning the notebook-size personal computer. This construction provides a stable placement of the notebook-size personal computer on the top of the body casing, so that the notebook-size personal computer is prevented from falling while the apparatus is used or transferred. Furthermore, the apparatus may preferably have a computer-fixing element with which the notebook-size personal computer can be fixed to or removed from the top of the body casing, which further improves the stability during the transfer.

### BRIEF DESCRIPTION OF THE DRAWINGS

FIG. 1 is an external view of a body composition measurement apparatus as an embodiment of the present invention.

FIG. 2 shows the electrical construction of the body composition measurement apparatus of the present embodiment.

FIG. 3A is an external view of a cable used in the present apparatus, FIG. 3B is a sectional view at line a-a′ in FIG. 3A, and FIG. 3C shows the sectional structure of a solid wire cable and a plug at a connection part.

FIG. 4 is a plan view of the top of the main unit of the present body composition measurement apparatus.

FIG. 5 is a front view of the main unit.

FIG. 6 is a side view of the main unit.

FIG. 7 is a front view of a personal computer placed on the main unit in the state of normal use.

FIG. 8 is a flowchart showing the measurement operation by the body composition measurement apparatus of the present embodiment.

FIG. 9 is a flowchart showing the measurement operation by the body composition measurement apparatus of the present embodiment.

FIG. 10 is an outlined drawing of an initial screen.

FIG. 11 is an outlined drawing of a body composition measurement screen.

FIG. 12 is a detailed drawing of a part of the screen shown in FIG. 11.

FIG. 13 is a detailed drawing of a part of the screen shown in FIG. 11.

FIG. 14 is a detailed drawing of a part of the screen shown in FIG. 11.

FIG. 15 is a detailed drawing of a part of the screen shown in FIG. 11.

FIG. 16 is a detailed drawing of a part of the screen shown in FIG. 11.

FIG. 17 is a detailed drawing of a part of the screen shown in FIG. 11.

FIG. 18 is a detailed drawing of a part of the screen shown in FIG. 11.

FIG. 19 is a detailed drawing of a part of the screen shown in FIG. 11.

FIG. 20 is a detailed drawing of a part of the screen shown in FIG. 11.

FIG. 21 shows an impedance model of the human body corresponding to the body composition measurement method used in the body composition measurement apparatus of the present embodiment.

FIG. 22A shows a model illustrating the state of acquisition of cross-sectional images with an MRI, and FIG. 22B is an example of a distribution chart of the mass of a tissue for each section of the body part.

FIG. 23A shows a composition model of a segment resulting from the division of the body, and FIG. 23B shows an equivalent circuit model of the tissues.

FIG. 24 shows a model of a measurement system in which the capacitance of the cable is considered.

FIG. 25 shows the electrical construction of the main part of a body composition measurement apparatus as another embodiment of the present invention.

### BEST MODE FOR CARRYING OUT THE INVENTION

Referring to the drawings, an embodiment of a body composition measurement apparatus using a body impedance measurement apparatus according to the present invention is described below.

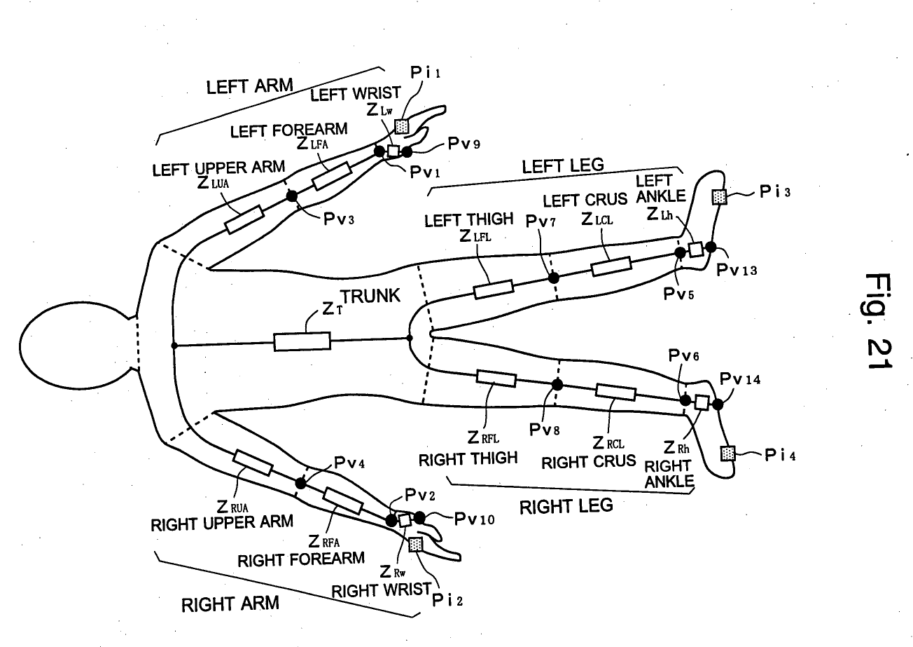

In advance of the description of the construction and operation of the body composition measurement apparatus of the present embodiment, the impedance measurement method used in the present body composition measurement apparatus is described. FIG. 21 shows an approximate model representing the impedance configuration of the human body corresponding to the present measurement method. In the apparatus of the present embodiment, the human body is divided into plural segments, and the impedance is measured for each segment. To improve the accuracy of estimation of the body composition information based on the impedance, the segment is defined corresponding to every part of the body where the composition of the tissue is relatively uniform, i.e. every part of the body that can be approximately represented by a columnar model, which will be explained later.

In detail, as shown in FIG. 21, the right and left arms (exclusive of the hands) are each divided at the elbow into the upper arm and the forearm, and the right and left legs (exclusive of the feet) are each divided at the knee into the thigh and the crus. Thus, the four limbs are divided into eight segments. In addition, the trunk (including the chest and the abdomen) is considered as one segment. The head and the fingertips of the hands and the feet are excluded from consideration for the present. Thus, the entire body is divided into nine segments. Each of the nine segments is independently assigned an impedance, and a model is created in which the impedances are connected as shown in FIG. 21. In FIG. 21, Z_{LFA}, Z_{LUA}, Z_{RFA}, Z_{RUA}, Z_{LFL}, Z_{LCL}, Z_{RFL}, Z_{RCL}, and Z_{T} denote the impedances of the left forearm, left upper arm, right forearm, right upper arm, left thigh, left crus, right thigh, right crus and trunk, respectively.

To measure the impedances of the nine segments, four current-supplying points (Pi₁ to Pi₄) and eight voltage-measuring points (Pv₁ to Pv₈) are determined on the limbs of the subject in a supine position. In this embodiment, the current-supplying points Pi₁ to Pi₄ are located in the vicinity of the root of the middle fingers on the backs of both hands and in the vicinity of the roots of the middle fingers on the insteps of both feet. The voltage-measuring points Pv₁ to Pv₈ are located at the right and left wrists, right and left elbows, right and left ankles and right and left knees. The voltage-measuring points Pv₁ and Pv₂ at the right and left wrists and Pv₅ and Pv₆ at the right and left ankles are relatively distant from the trunk. Therefore, the measurement of voltage using these four voltage-measuring points is called the “distal measurement” hereinafter. On the other hand, the voltage-measuring points Pv₃ and Pv₄ at the right and left elbows and Pv₇ and Pv₈ at the right and left knees are relatively close to the trunk. Therefore, the measurement of voltage using these four voltage-measuring points is called the “proximal measurement” hereinafter.

When a radio frequency current is supplied between the two points selected from the four current-supplying points Pi₁ to Pi₄, the potential difference between a predetermined pair of the voltage-measuring points can be regarded as a potential difference generated between both ends of an impedance, or between both ends of plural impedances connected in series. In this case, the current barely flows through such parts of the body that are not on the current path. Therefore, it is possible to regard such parts as mere lead wires pulled out from both ends of the impedance of the target part, ignoring their impedances.

Suppose the current is supplied between the current-supplying points Pi₁ and Pi₂ located at both hands. In this case, the potential difference between the voltage-measuring points Pv₁ and Pv₂ located at both wrists (i.e. distal measurement) is equal to the voltage corresponding to the impedance composed of Z_{LFA}, Z_{LUA}, Z_{RFA} and Z_{RUA} connected in series; i.e. the impedance of the right and left arms. The potential difference between the voltage-measuring points Pv₃ and Pv₄ located at both elbows (i.e. proximal measurement) is equal to the voltage corresponding to the impedance Z_{LUA} and Z_{RUA} connected in series; i.e. the impedance of the right and left upper arms. The potential difference between the voltage-measuring points Pv₁ located at the left wrist and Pv₅ located at the left ankle (or Pv₆ located at the right ankle) is equal to the voltage corresponding to the impedance Z_{LFA} and Z_{LUA} connected in series, i.e. the impedance of the left arm, because the right and left legs and the trunk can be regarded as mere lead wires. The potential difference between the voltage-measuring points Pv₃ located at the left elbow and Pv₇ located at the left knee (or Pv₈ located at the right knee) is equal to the voltage corresponding to the impedance Z_{LUA}, i.e. the impedance of the left upper arm, because the right and left thighs and the trunk can be regarded as mere lead wires.

The measurement can be similarly performed for other parts of the body. These measurement results make it possible to accurately measure the impedance of each of the nine segments. Based on the measurement values of the impedance, or based on the measurement value of the impedance and body specific information, the body composition information is estimated. The estimation is performed by, for example, using an estimation formula prepared using body composition information collected with an MRI.

In the body composition measurement apparatus of the present embodiment, the accuracy of the estimation formula itself is enhanced by performing MRI measurements for a number of monitors with different body specific information including the height, weight, age, gender, etc., and deriving reliable regression analysis coefficients from the results of the measurements. An example of the method of estimating the body composition information is as follows.

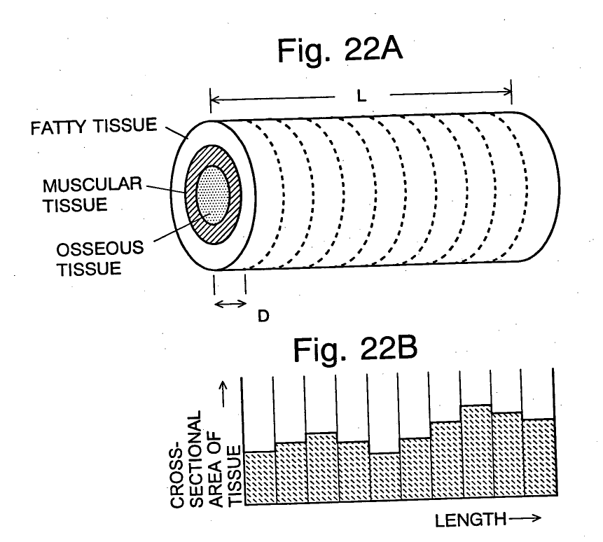

An MRI is capable of acquiring cross-sectional images of any part of the human body. The cross-sectional images provide information about the masses and/or ratios of the tissues, such as muscle, fat and bone, within the cross section. FIG. 22A shows cross-sectional images acquired for each thickness D along the longitudinal direction of a body part. From these images, the masses (or areas) of the tissues, such as muscle, fat and bone, are calculated. From this calculation, the area distribution of the tissues along the longitudinal direction of the body part can be obtained, as shown in FIG. 22B. Then, the areas are integrated along the longitudinal direction to determine the masses of the tissues within the body part concerned. In the present measurement method, the body is divided into nine segments, so that it is easy to perform the MRI measurement for each unitary segment. Furthermore, since each segment is defined so that it approximates a columnar body, the mass of each tissue can be obtained with high accuracy.

In regard to the method of estimating body composition for each segment, an example of the method of estimating the lean body mass is described below.

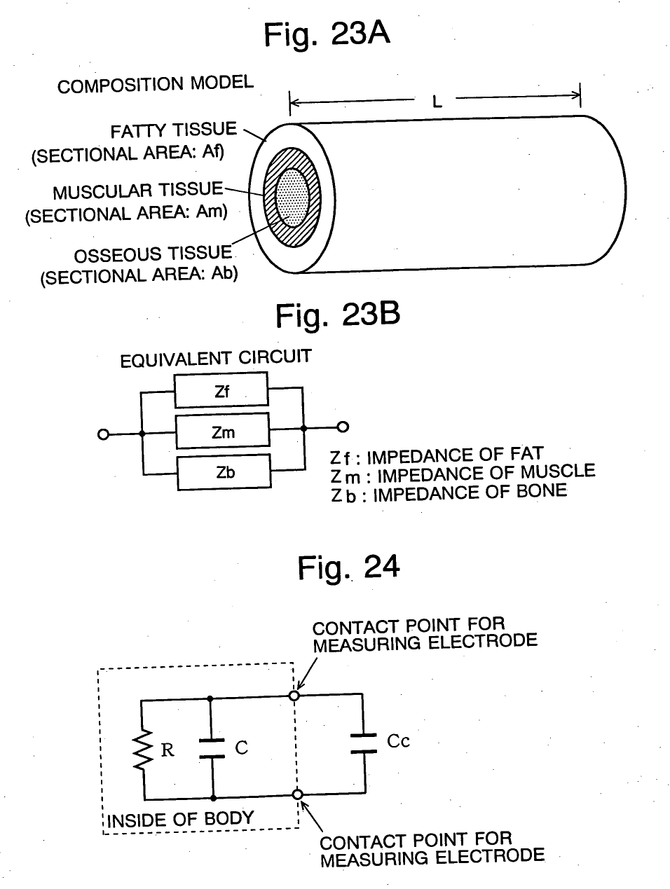

Columnar composition model as shown in FIG. 23A is applied to each of the nine segments. Each segment is assumed to contain the fatty tissue having cross-sectional area A_{f}, muscular tissue having cross-sectional area Aₘ and osseous tissue having cross-sectional area A_{b}, all tissues having the same length L. Denoting the volume resistivity of the fatty tissue, muscular tissue and osseous tissue as ρ_{f}, ρₘ and ρ_{b}, respectively, the impedance Z_{f}, Zₘ and Z_{b} of the fatty tissue, muscular tissue and osseous tissue are given as follows:
Z_{f}=ρ_{f}×(L/A_{f}),
Zₘ=ρₘ×(L/Aₘ),
Z_{b}=ρ_{b}×(L/A_{b}).
The impedance Z₀ of the entire segment can be approximately represented by a model composed of the impedances Z_{f}, Zₘ and Z_{b} connected in parallel, as shown in FIG. 23B. Accordingly, the impedance Z₀ is given as follows:
1/Z₀=(1/Z_{f})+(1/Zₘ)+(1/Z_{b}) (1).

Now, the volume and density of non-fat layer (the layer other than the fat layer or panniculus) are denoted by V_{LBM} and D_{LBM}. The density D_{LBM} is known from conventional studies. Then, the lean body mass (LBM) is given by:

$$\begin{matrix}LBM = {V}_{LBM} \times {D}_{LBM} , & \\ where & \\ {V}_{LBM} = {A}_{LBM} \times L & ( 2 ) \\ = ( {A}_{m} + {A}_{b} ) \times L & \\ = {ρ}_{m} \times ( {L}^{2} / {Z}_{m} ) + {ρ}_{b} \times ( {L}^{2} / {Z}_{b} ) . & \end{matrix}$$

Substituting formula (1) into formula (2),
V_{LBM}=ρₘ×L²×[1/Z₀−1/Z_{f}]+(ρ_{b}−ρₘ)×(L²/Z_{b}) (3),
where the volume resistivities of the tissues satisfy the condition: ρₘ<ρ_{b}<<ρ_{f}.

It is assumed here that there is no influence from distal parts such as wrists or ankles (Condition A). Then
A_{b}<<Aₘ.
Therefore,
Z_{f}(=ρ_{f}×L/A_{f})>Z_{b}(=ρ_{b}×L/A_{b})>>Zₘ(=ρₘ×L/Aₘ)>Z₀.
Applying this to formula (3),
V_{LBM}=ρₘ×(L²/Z₀)+(ρ_{b}−ρₘ)×(L²/Z_{b}) (4).
Here,
ρₘ×(L ²/Z₀)>>(ρ_{b}−ρₘ)×(L²/Z_{b}),
so that
V_{LBM}=ρₘ×(L²/Z₀),
and
LBM=D_{LBM}×ρₘ×(L²/Z₀).
Therefore, using a predetermined function f(x), the following relation is obtained:
LBM=f(L²/Z₀).

Now, the influences from distal parts, such as wrists or ankles, are taken into account (Condition B). In this case,
A_{b}<Aₘ.
Therefore
ρₘ×(L²/Z₀)>(ρ_{b}−ρₘ)×(L²/Z_{b})=ΔV_{b}.
In general, the heavier the body is, the greater the volume V_{b} of the osseous tissue becomes to support the body. Accordingly, the following relation can be assumed: V_{b}∝ΔV_{b}∝f(W). Therefore, from formula (4),

$$\begin{matrix}{V}_{LBM} = {ρ}_{m} \times ( {L}^{2} / {Z}_{0} ) + ( {ρ}_{b} - {ρ}_{m} ) \times ( {L}^{2} / {Z}_{b} ) \\ = {ρ}_{m} \times ( {L}^{2} / {Z}_{0} ) + Δ {V}_{b} \\ \approx {ρ}_{m} \times ( {L}^{2} / {Z}_{0} ) + f ( W ) , \\ so that \\ LBM = f ( {L}^{2} / {Z}_{0} , W ) .\end{matrix}$$

Taking into account the change of tissues with aging and the difference depending on genders, the estimation formula for multiple regression analysis can be created as follows:
LBM=a″+b″×(L²/Z₀)+c″W+d″×Ag (5),
where a″, b″, c″ and d″ are constants (multiple regression coefficients), which take different values for different genders. The values of a″, b″, c″ and d″ for each gender can be determined beforehand by applying the lean body mass (LBM) obtained by an MRI method to the estimation formula for multiple regression analysis.

An example of the method of estimating the muscle mass is as follows. This is basically the same as the above-described estimation of lean body mass. Denoting the volume and density of the muscle layer as V_{MM} and D_{MM}, respectively, the muscle mass MM is given by:
MM=V_{MM}×D_{MM}.
Using the impedance Zₘ of the muscle layer,
V_{MM}=ρₘ×(L²/Zₘ).

Under the condition A,
MM≈LBM=a+b×(L²/Z₀)+c×Ag (6).
Under the condition B, on the other hand,

$$\begin{matrix}LBM = MM + BM \\ = a + b \times ( {L}^{2} / {Z}_{0} ) + c \times W + d \times Ag .\end{matrix} \qquad ( 7 )$$

In formula (7), the term L²/Z₀ contains information about bone (BM) in addition to the muscle mass (MM); it is impossible to separate them. Among the nine segments, upper arms and thighs satisfy the condition A, and forearms and crura satisfy the condition B.

It is known that, for each person, the muscle mass of the upper arm is highly correlated with that of the forearm, and the muscle mass of the thigh is highly correlated with that of the crus. Accordingly, information about muscle mass of the upper arm (MM_{U}) and information about muscle mass of the forearm (MM_{F}) are obtained as follows. That is, based on the regression analysis of MM_{UA} and MM_{FA} obtained by an MRI method, the following estimation formula is created:
MM_{FA}=aₘ+bₘ×MM_{UA} (8).

Similarly, muscle mass of the crus (MM_{CL}) is estimated from the information about the muscle mass of the thigh (MM_{FL}) obtained by an MRI method:
MM_{CL}=a′ₘ+b′ₘ×MM_{FL} (9).

The muscle mass of a proximal segment, such as the upper arm or thigh, can be obtained by formula (6) because it satisfies the condition A. Further, by substituting the muscle masses of the upper arm and the thigh, obtained by formula (6), into formula (8) and (19), the muscle masses of the forearm that of the crus can be estimated.

The bone mass of each segment can be similarly calculated.

To estimate the lean body mass, muscle mass and bone mass of the entire body, one method is to estimate the body composition for each unitary segment and incorporate the estimated values into a formula for estimating the body composition of the entire body. Another method is to create a regression formula with the unitary segments of the four limbs and the trunk used as independent variables. As regards the method of estimating information about body composition and health conditions using the measurement values of the impedance and the body specific information, the methods proposed by the applicant in the Japanese Patent Application No. 2000-362896 may be used, or other methods may be used.

Next, the construction and operation of the body composition measurement apparatus of the present embodiment is described.

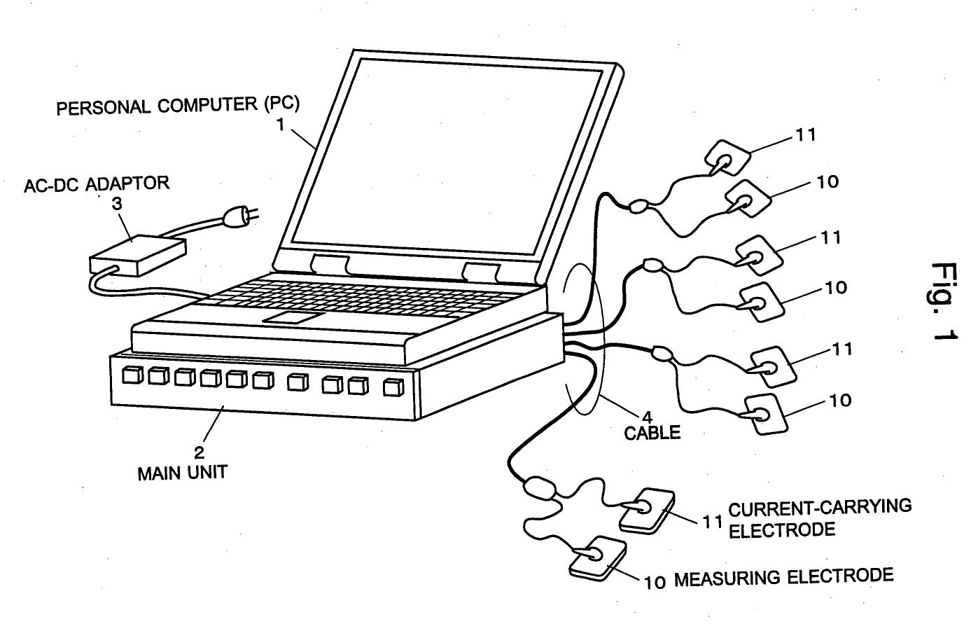

FIG. 1 shows the external of the body composition measurement apparatus of the present embodiment. The present body composition measurement apparatus is constructed to supply a weak radio frequency current through the body of the subject, to detect the voltage generated by the current in a predetermined part of the body, to calculate the impedance from the voltage value and the current value, to perform a calculation wherein the measurement value of the impedance and the body specific information, such as the height, weight, age and gender, which have been externally entered, are applied to a predetermined estimation formula, and to calculate and show the body composition information (such as body-fat ratio, lean body mass, body-fat mass, total body water, muscle mass, muscle force, bone mass, bone density, degree of obesity, basal metabolic rate and ADL index) and health condition information of the subject.

As shown in FIG. 1, the present body composition measurement apparatus includes a notebook-size personal computer (which is referred to as the “PC” hereinafter) 1 used mainly as a controller and data processor, and a main unit 2 used mainly for measuring impedances. Plural electrodes necessary for the measurement are taken out from the back of the main unit 2 via cables 4. The power cable for taking commercial AC power supply is connected to the main unit 2 via an AC-DC adapter 3.

The electrodes include current-supplying electrodes 10 for supplying the current and measuring electrodes 11 for measuring the voltage. One current-carrying electrode 10 is paired with one measuring electrode 11, and the pair is connected via a low-inductive cable 4 to the main unit 2. The current-carrying electrodes 10 and the measuring electrodes 11 can be securely and immovably attached to the skin of the subject. Each electrode is designed to have a flat affix-type body so as to decrease the impedance, or contact resistance, of the electrode itself.

The present body composition measurement apparatus is designed to perform the impedance measurement by using four current-carrying electrodes 10 and four measuring electrodes 11, paired with each other. When, as will be described later, the measurement should be performed for eight voltage-measuring points, the examiner should rearrange the measuring electrodes 11 on the body of the subject after every measurement for every four points. This is because use of a large number of electrodes would not only increase the production costs of the apparatus, but also makes the preparation for the measurement more troublesome due to the entanglement of the cables. Furthermore, mistaken attachments of electrodes to the subject would be more probable. In the case where these problems do not matter, the apparatus may be designed to have eight or sixteen measuring electrodes.

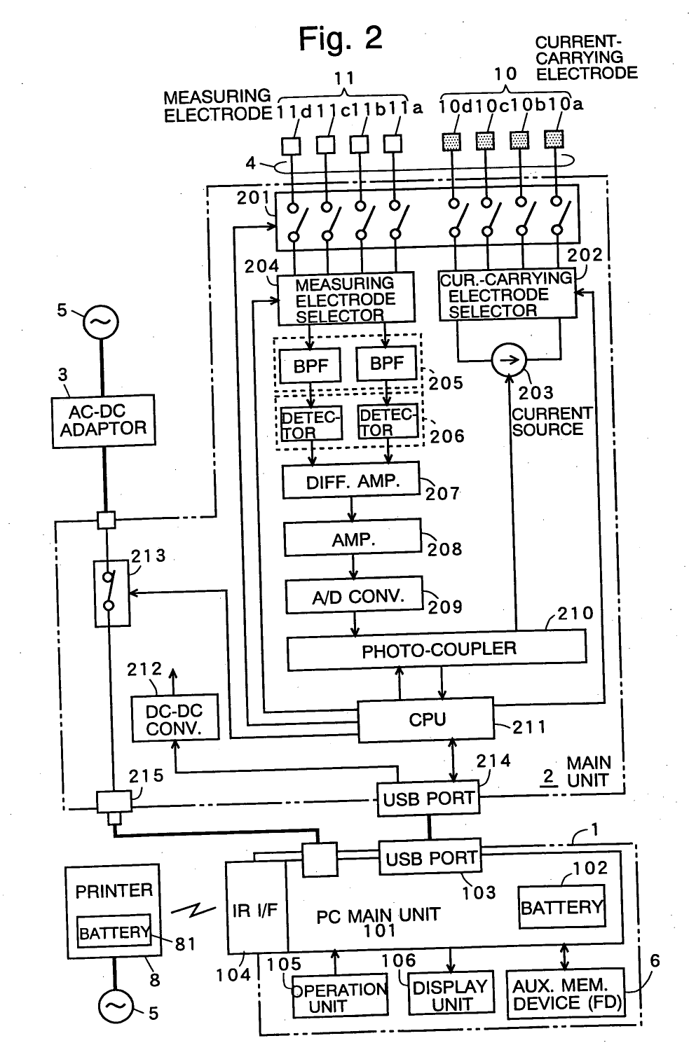

FIG. 2 shows the electrical construction of the present body composition measurement apparatus. Four current-carrying electrodes 10a, 10b, 10c and 10d are connected via signal line opening/closing relays 201 to a current-carrying electrode selector 202, which selects two electrodes to be connected to a current source 203. The current source 203 generates a constant radio frequency current at frequency f₀, which is usually determined within the range 10 kHz to 100 kHz. Similarly, the four measuring electrodes 11a, 11b, 11c and 11d are connected via the signal line opening/closing relays 201 to a measuring electrode selector 204, which selects two electrodes and transfers the signals obtained with each of the two electrodes to each of band-pass filters (BPF) 205.

The band-pass filters 205 remove component signals other than the frequency f₀. After that, detectors 206 detect and rectify the signals to extract component signals of frequency f₀. The signals detected in parallel are differentially amplified by a differential amplifier 207, and then further amplified by an amplifier 208. Analogue-to-digital converter (A/D) 209 converts the signals into digital signals, and sends the digital signals via a photo-coupler 210 to a central processing unit (CPU) 211. The CPU 211 is connected to the universal serial bus (USB) port 214 and has the function of the conversion/inversion of data for the USB interface. The CPU 211 not only sends to the USB terminal 214 the data corresponding to the output signal of the A/D converter 209, but also receives control signals through the USB port 214 and, based on the control signals, controls the operation of the current source 203 and the operations of the signal line opening/closing relays 201 and a power line opening/closing relay 213 (which will be mentioned later) through the photo-coupler 210. The optical connection between the CPU 211 and the analogue measurement circuits via the photo-coupler 210 prevents the intrusion of digital noises generated by the CPU 211 or coming from the PC 1 into the analogue measurement circuits.

The DC power output of the AC-DC adaptor 3 connected to a commercial AC power supply 5 is drawn into the main unit 2 and connected via the power line opening/closing relay 213 to a power output jack 215. The power cable for supplying electric power to the PC 1 is connected to the power output jack 205. Thus, the DC power output of the AC-DC adaptor 3 is connected to the PC 1, where the main unit 2 merely provides a pathway having only the power line opening/closing relay 213.

The PC 1 has a main unit 101 enclosing the CPU, read only memory (ROM), random access memory (RAM), hard disk drive, battery 102 and other components, an operation unit 105 including a keyboard and a pointing device, such as a mouse, a display unit 106 composed of a liquid crystal display, and an auxiliary storage device 6 such as a floppy-disk drive (FD). Furthermore, the PC 1 has an infrared interface (I/F) 104 for connection with a printer 8. This construction includes no electrical connection by cables, so that the influence from the noises generated by the power supply of the printer 8 is eliminated. Furthermore, even if parts trouble or other trouble has occurred, the construction prevents an excessive current from the printer 8. Thus, such a case is assuredly avoided where an abnormal current flows through the body of the subject. The printer 8 has a battery 81 of its own so that the entire apparatus including the printer 8 can be driven by the battery.

The PC 1 has a standard USB port 103. As is generally known, USB interface is provided with lines for transferring serial data and DC power. In this embodiment, the USB port 103 of the PC 1 is capable of providing a maximum power output of 5V/500 mA. The main unit 2, connected to the PC 1 via a USB cable, receives the DC power from the PC 1 and distributes the power to each circuit through a DC-DC converter 212. Accordingly, every electrical circuit included in the main unit 2 is designed to operate by the power of 5V/500 mA or lower. In addition, the DC-DC converter 212 prevents the intrusion of noises through the power source into the analogue measurement circuit.

A calculation program for measuring the impedance and performing the calculations to estimate various kinds of information relating to the aforementioned body composition information and health condition based on the measurement value of the impedance, and a control program for conducting the measurement, are stored on the hard disk of the PC 1. The program is executed in response to a command externally given through the operation unit 105, whereby the measurement of the impedance (to be described later) followed by calculation and display processes is practically performed.

The present body composition measurement apparatus has such features that the signal line opening/closing relay 201 is provided for each cable 4 (i.e. signal path) connected to the current-carrying electrodes 10 and the measuring electrodes 11, and that the power line opening/closing relay 213 is provided for the power supply line connected via the AC-DC adaptor 3 to the commercial AC power supply 5. The function of the signal line opening/closing relays 201 is to substantially disconnect the electrodes 10 and 11 from the main unit 2 at all times except for the period when the impedance of the body of the subject is measured. This prevents an undesirable current from flowing through the body of the subject when a trouble or malfunctioning of the circuits has occurred, thus ensuring the safety of the subject.

The function of the power line opening/closing relay 213, on the other hand, is to disconnect the commercial AC power supply 5 from the main unit 2 and the PC 1 during the measurement of the impedance, to block the noises coming from the outside through the commercial AC power supply 5. This suppresses the noises during the measurement of the impedance, whereby the accuracy of the measurement is improved. Another function is to disconnect the commercial AC power supply 5 during the process of connecting the measurement circuits and the body via the electrodes 10 and 11 so that at least a leakage of the alternating current of 100V is prevented from entering the body when a trouble or malfunctioning of the circuits has occurred. This constitutes a dual safety measure with the signal line opening/closing relays 201. The measurement operation will be detailed later.

In the present body composition measurement apparatus, the cable 4 pulled out from the main unit 2 must have a considerable length so that the electrode can be attached to various parts of the body, as described above. In general, a long-pulled cable functions as an antenna and may pick up external induction noise. For accurate measurement, such induction noise must be suppressed to the utmost. The cable 4 (or, exactly, signal path) has a capacitance of its own, such as parasitic capacitance. If this capacitance is mistaken for an impedance of the body, the accuracy of the measurement becomes lower. In view of the above problems, the apparatus of the present embodiment has adopted a special structure designed for suppressing the induction noise and the influence from the capacitance of the cable itself.

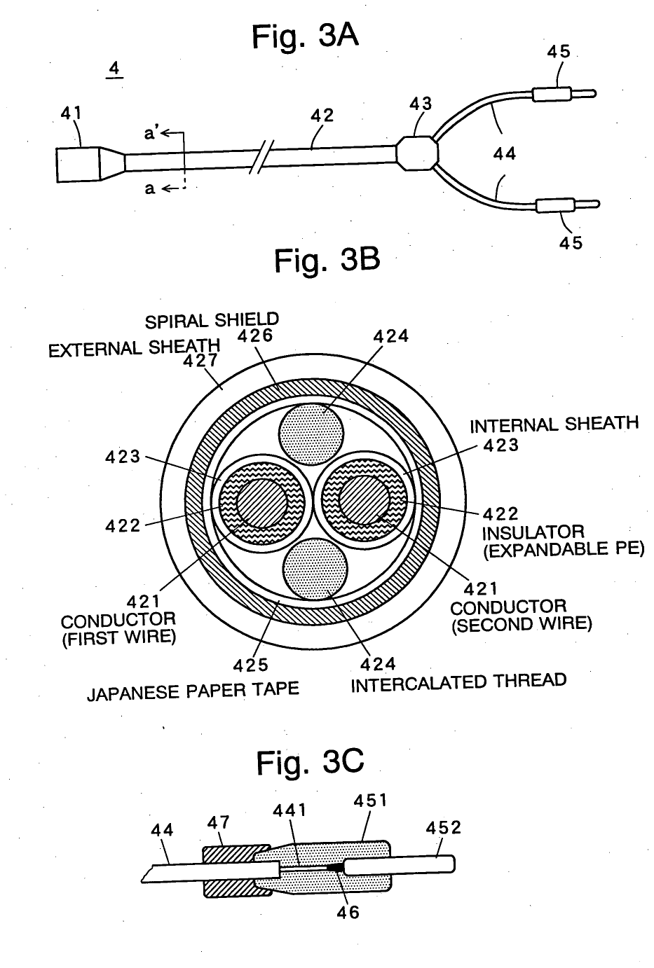

FIG. 3A is an external view of the cable 4 used in the present apparatus, and FIG. 3B is a cross-sectional view at line a-a′ in FIG. 3A. As shown in FIG. 3A, the cable 4 includes a balanced type shield cable 42 having a straight plug 41 to be connected to the main unit 2 at one end and a branch mold 43 branching into single wire cables 44 connected to the two wires, respectively. The single wire cable 44 has, at its end, a plug 45 to which an electrode is fixed. The connection part between the single wire cable 44 and the plug 45 has a cross-sectional structure as shown in FIG. 3C, where the wire 441 of the single wire cable 44 is soldered to the conductive bar 452 of the plug 45 (at the point 46). The soldered part is embedded in the resin housing 451 of the plug 45. To prevent the single wire cable 44 from rotating and breaking the soldered part, the housing 451 and the single wire cable 44 are bound together by a heat-shrinkable tubing 47 put across their boundary. Molding with bonding agent is also usable in place of the heat-shrinkable tubing.

As shown in FIG. 3B, the balanced type shield wire is composed of conductors 421 as the first and second wires, each surrounded by an internal sheath 423, with an insulator 422 made of expandable polyethylene resin having an expansion ratio of 75 to 80% filled in the internal space. Two pieces of such structures are bundled together with two pieces of intercalated threads 424, which are enveloped by a Japanese paper tape 425. This structure is then covered by a cylindrical spiral shield 426, and further by an external sheath 427. In the present apparatus, the cable is about 200 cm in length, and the balanced type shield cable is about 160 cm in length. The four cables are all identical in these lengths.

The above structure not only suppresses the capacitance of the cable but also reduces the difference between plural cables. Furthermore, it suppresses the change in capacitance due to the bending stress or tensile stress. In concrete, the following properties are realized.

Cable capacitance (at frequency of 50 kHz): 100 pF or lower.

Change in capacitance due to bending: within ±10 pF (change in capacitance observed when a bending test is performed twenty times for each of three points in the cable, with the tensile load 300 g and the bending angle ±60 degrees).

Change in capacitance due to tensile stress: within ±10 pF (change in capacitance observed when the cable is maintained under the tensile load of 500 g for 60 seconds).

One current-carrying electrode and one measuring electrode to be attached to the same part of the upper or lower limb are paired, and the first and second wires of the same cable are used as the signal paths leading to the paired electrodes. For example, if the first wire of a cable constitutes the signal path leading to the current-carrying electrode for right hand, then the second wire of the cable constitutes the signal path leading to the measuring electrode for right hand. Thus, the signal paths leading to the four measuring electrodes 11a-11d are contained in the shields of different cables. This reduces the capacitance between two measuring electrodes observed during the measurement of the impedance, and suppresses the influence from the noise during the measurement of the impedance.

The shield wires of the four cables are short-circuited at a point near the entrance of the main unit 2 so that they have almost the same earth potential. This provides such an effect that the total capacitance of the cables 4 added in parallel to the body of the subject is maintained almost unchanged when the measuring electrode selector 204 is switched so that two measuring electrodes and the cables leading to these measuring electrodes are connected to the measuring circuit located behind them.

It should be noted that the use of expandable polyethylene resin with an expansion ratio of 75 to 80% as the insulator 422 of this embodiment is a mere example.

In general, the capacitance C of a balanced type cable is given by the following formula:
C=12.08×εₑ/Log₁₀(1.2×B/K1×D)[pF/m],
where B is the distance [mm] between the conductors, K1 is the coefficient for effective radius of internal conductor, and D is the external diameter [mm] of the conductor. This shows that the capacitance C is proportional to the effective specific inductive capacity εₑ. Here,
εₑ=ε_{A}^{1-V}
where ε_{A} is the specific inductive capacity of the dielectric and V is the expansion ratio (i.e. occupation ratio of air). Therefore, the capacitance C can be decreased by using a material having a low specific inductive capacity and increasing the expansion ratio. However, the higher the expansion ratio is, the lower the stress resistance or other property is. In view of the above conditions, the material to use and its expansion ratio should be determined.

In the body composition measurement apparatus of the present embodiment, the capacitance of the cable 4 itself is suppressed as described above, and the intrusion of the inductive noise is decreased. However, the capacitance of the cable 4 cannot be completely zero. Furthermore, to perform a very accurate measurement, it is necessary to consider the capacitance and other properties of analogue switches used in the measuring electrode selector 204 in addition to the cables 4. Accordingly, the body composition measurement apparatus of the present embodiment is constructed to perform a correction in the process of calculating the impedance, to eliminate the aforementioned influence from the capacitance of the cable 4 and/or the analogue switches.

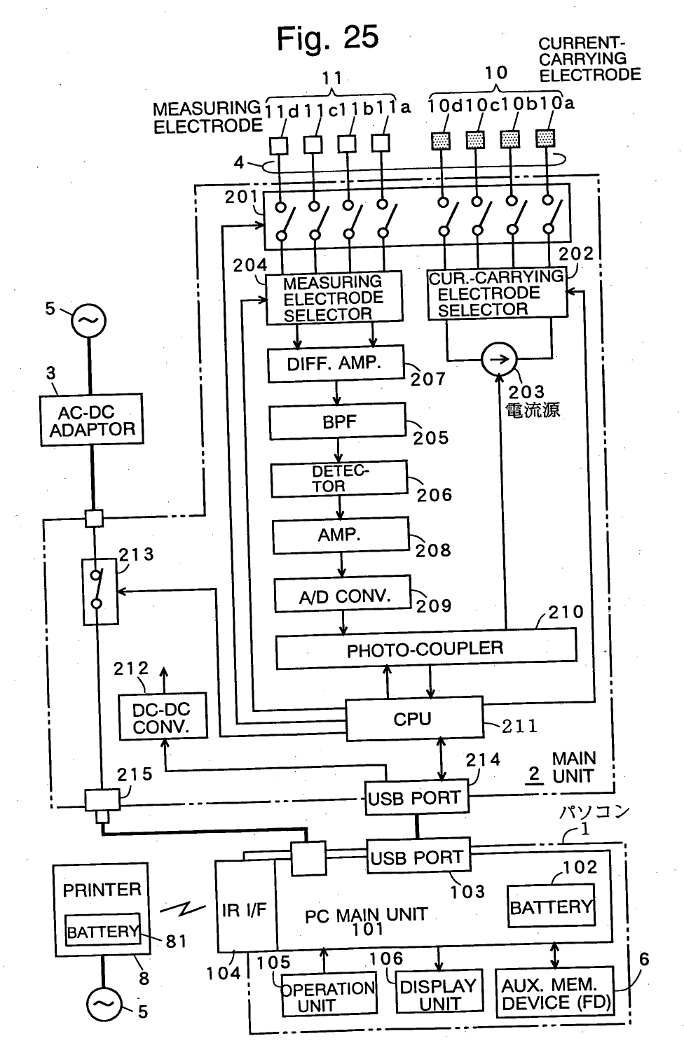

The process of correcting the capacitance is described below. The following discussion uses the model shown in FIG. 24, in which the cable is considered. In practice, the capacitance and other properties of the analogue switches should be considered in addition to the cable, as explained above, and they are generally referred to as the “cable capacitance” here. According to the model in FIG. 24, the impedance Zₘ obtained by the measurement consists of the bioelectric impedance Z₀ and the cable capacitance C_{c} connected in parallel.

In the present model, Zₘ, Z₀, R, C and C_{c} suffice the following relations:

$$\begin{matrix}{Z}_{0} = \frac{R}{\sqrt{1 + {( ω \cdot C \cdot R )}^{2}}} , & ( 11 ) \\ {Z}_{m} = \frac{R}{\sqrt{1 + {[ ω \cdot ( C + {C}_{c} ) \cdot R ]}^{2}}} . & ( 12 )\end{matrix}$$

Using the notation Cₐ=C+C_{c}, formula (12) can be rewritten as follows:

$${Z}_{m} = \frac{R}{\sqrt{1 + {[ ω \cdot {C}_{a} \cdot R ]}^{2}}} . \qquad ( 13 )$$

From formula (11), the following formula is obtained:

$${Z}_{0}^{2} = \frac{{R}^{2}}{1 + {( ω \cdot C \cdot R )}^{2}} . \qquad ( 14 )$$

From formula (12),

$${Z}_{m}^{2} = \frac{{R}^{2}}{1 + {( ω \cdot {C}_{a} \cdot R )}^{2}} .$$

This can be rewritten as follows:

$${R}^{2} = \frac{{Z}_{m}^{2}}{1 - {( ω \cdot {C}_{a} \cdot {Z}_{m} )}^{2}} . \qquad ( 15 )$$

Substituting formula (15) into formula (14),

$${Z}_{0}^{2} = \frac{{Z}_{m}^{2}}{1 - {( ω \cdot {Z}_{m} )}^{2} \cdot ( {C}_{a}^{2} - {C}^{2} )} .$$

Therefore, Z₀ is given by:

$${Z}_{0} = \frac{{Z}_{m}}{\sqrt{1 - {( ω \cdot {Z}_{m} )}^{2} \cdot ( {C}_{a}^{2} - {C}^{2} )}} . \qquad ( 16 )$$

Formula (16) is the correction formula, which removes the influence of the cable capacitance C_{c} from the measurement value Zₘ of the impedance obtained as a result of measuring the subject, to calculate the bioelectric impedance Z₀. The value C used here is the average of values obtained beforehand by experimental measurements. It is assumed here that the value is constant regardless of body parts. The cable capacitance C_{c} can be measured beforehand by connecting a condenser and a resistance, each having a known value, to both ends of the cable 4 and measuring the impedance with an impedance meter or similar apparatus.

In practice, the above correction formula corrects not only the capacitance of the cable 4; it also corrects the capacitance observed from the body in FIG. 24, i.e. the entire input capacitance of the apparatus present between the two measuring electrodes 11. This capacitance includes the capacitance of the cable, the input capacitance of the analogue switches (the measuring electrode selector 204 in FIG. 2), the input capacitance of, for example, the operational amplifier or other elements of the band-pass filter 205, etc. The cable 4 has a resistance in addition to the capacitance. However, the resistance constitutes only a small portion of the impedance, so that it can be ignored in practice. It is of course possible to consider the resistance when creating the correction formula.

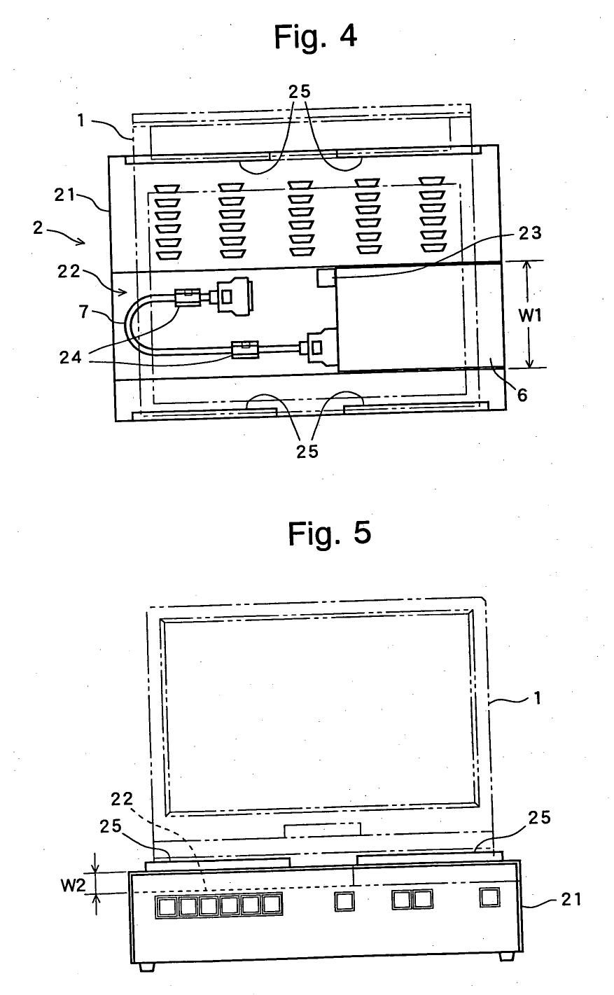

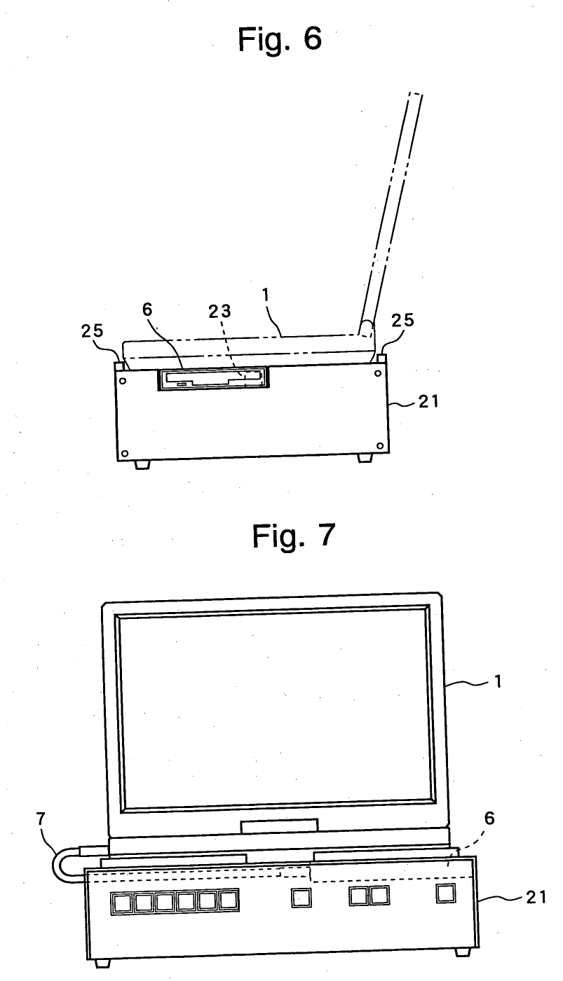

The body composition measurement apparatus of the present embodiment is also characterized by its external structure. FIG. 4 is a plan view showing the top of the main unit 2 of the present body composition measurement apparatus, FIG. 5 is a front view of the main unit 2, FIG. 6 is a side view of the main unit, and FIG. 7 is a front view of the PC 1 placed on the main unit 2 in the state of normal use.

One of the features in the external structure of the body composition measurement apparatus of this embodiment is that the square-shaped body casing 21 of the main unit 2 has a recess 22 laterally extending on its top. The width W1 of the recess 22 in the length direction is slightly larger than the width of the external floppy-disk drive 6 of the PC 1, and the depth W2 of the recess 22 is slightly larger than the height of the floppy-disk drive 6. Therefore, the floppy-disk drive 6 can be completely contained in the recess 22, as shown in FIG. 6. At the bottom of the recess 22, there is a stopper 23 for determining the backmost position of the floppy-disk drive 6. When the floppy-disk drive 6 is set in the recess 22 with its back-end in contact with the stopper 23, the front face of the floppy-disk drive 6 forms an almost flat surface with the side face of the body casing 21 of the main unit 2. Furthermore, at the bottom of the recess 22, there are holders 24 for holding the cable 7 for connecting the floppy-disk drive 6 and the PC 1. The holders 24 are designed to hold the cable 7 at two points close to both ends. When held by the holders 24, the cable 7 is entirely contained in the recess 22, as shown in FIG. 4. The body casing 21 also has stoppers 25 at the front and rear edges of its top, respectively. When the PC 1 is placed on the top, the stoppers 25 determine the position of the PC 1 in the length direction.

As described above, the floppy-disk drive 6 can be completely contained in the recess 22 without protruding from the top of the body casing 21, so that the PC 1 can be stably placed on the top. To use the floppy-disk drive 6, the cable 7 should be detached from the holder 24 holding an end of the cable 7 and connected to a terminal of the PC 1, as shown in FIG. 7. When, for example, the user pushes the front side of the floppy-disk drive 6 with a finger to insert or remove a floppy disk, the floppy-disk drive 6 does not move backward because the stopper 23 prevents its backward movement.

Thus, in the body composition measurement apparatus of this embodiment, an external floppy-disk drive of the PC 1 can be contained in a part of the body casing of the main unit 2, and the apparatus can be used in this state. This reduces the occupation space, and improves the user-friendliness. The PC 1 and the floppy-disk drive 6 are placed on the main unit 2 to constitute a unit, where, especially, the back-and-forth movement of the PC 1 prevented by the stoppers 25. Therefore, the user can carry the entire unit by holding only the main unit 2.

In the electric construction shown in FIG. 2, the band-pass filters 205 and the detectors 206 are placed before the differential amplifier 207. This construction requires these circuits to be provided for each of the two input lines. Instead, the construction shown in FIG. 25 may be adopted, where the band-pass filter 205 and the detector 206 are placed behind the differential amplifier 207. This construction is advantageous in that there is little influence from the noise, because the differential amplifier 207 cancels the common mode noise. The construction shown in FIG. 2, on the other hand, is advantageous in that it reduces the measurement error, because it is hardly affected by the stray capacitance of the cables and the circuits, and it allows only a small phase rotation even when the two loads connected to the inputs of the band-pass filters 205 via the measuring electrodes is unbalanced.

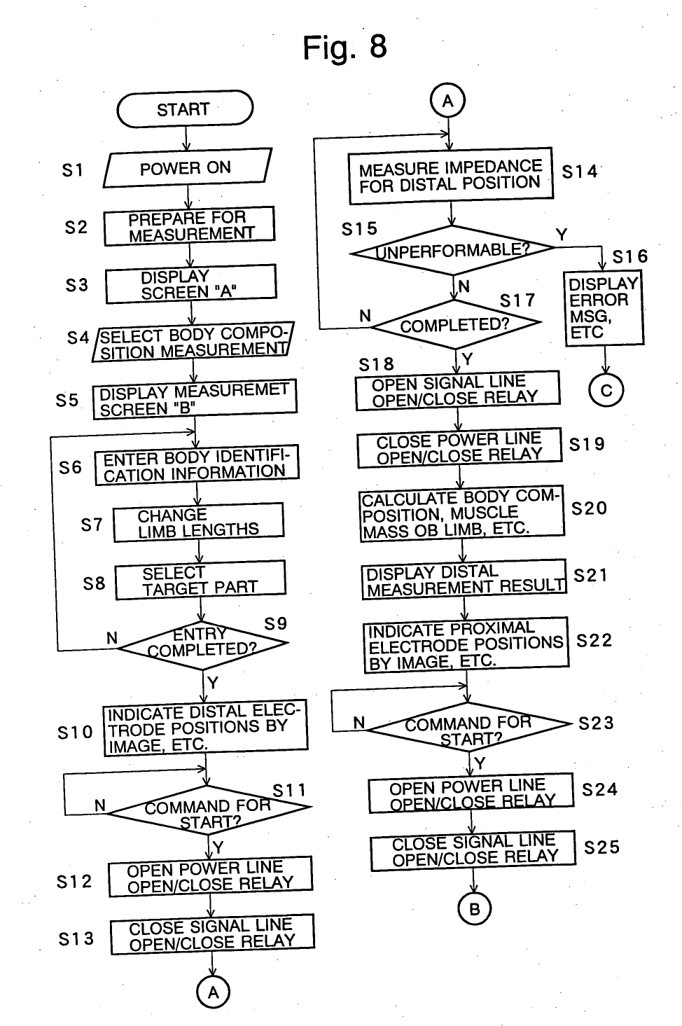

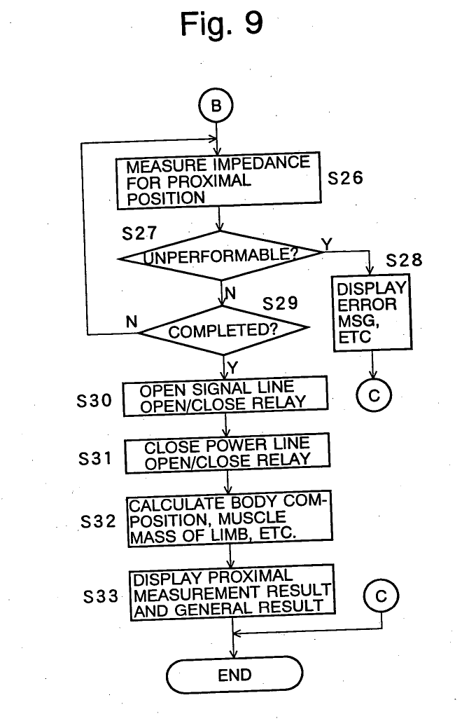

A practical example of the measurement operation performed by the present body composition measurement apparatus is described below. FIGS. 8 and 9 are flowcharts showing an operation for measuring the impedance for each of the aforementioned nine segments and estimating the body composition information from the measurement values of the impedance.

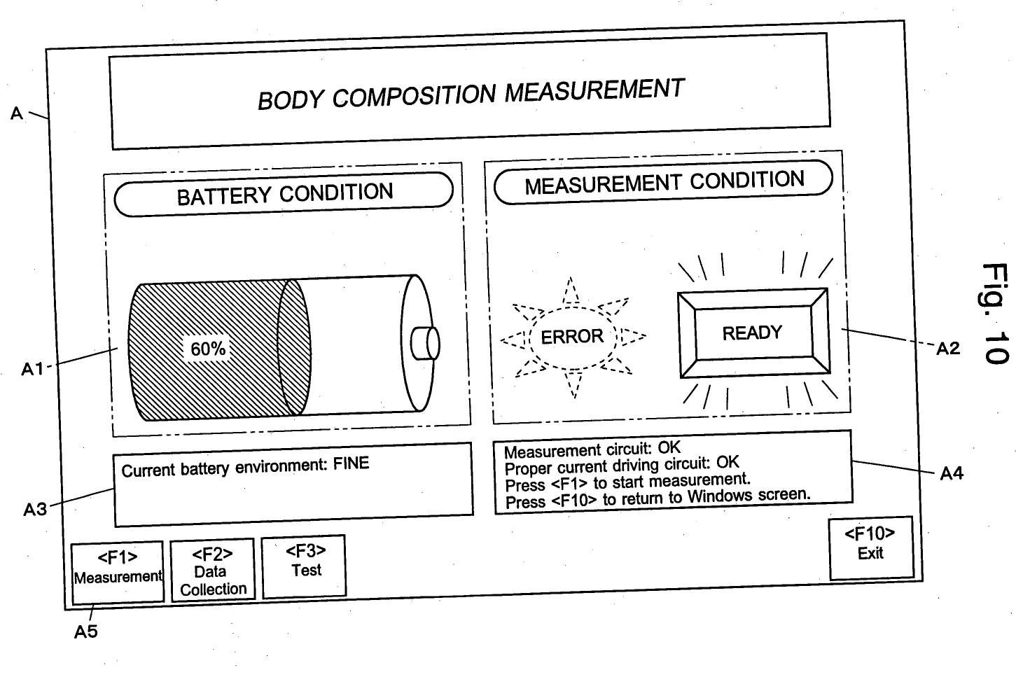

When the examiner or other person turns on the power switch of the PC 1 (Step S1), the PC 1 starts running and performs a measurement preparation process including initialization, checking of the remaining power of the battery 102, self-checking of the measurement circuits, etc (Step S2). After the measurement preparation process, an initial screen “A” as shown in FIG. 10 is displayed on the display unit 106 (Step S3).

The initial screen A has a battery power indicator A1 with a battery mark image similar to a cell, and a message display area A3 for showing text messages about the states of the remaining battery power. These components show the remaining battery power by the area and color of the painted part of the battery mark image, the numerical value, etc. When the remaining battery power is not sufficient, a message for prompting the charging of the battery or similar message is displayed. The initial screen A also includes a measurement circuit check result display area A2 for showing the result of the checking of the measurement circuits, and a message display area A4 for showing the result by text messages. These components provide information about whether any abnormality has been detected by the measurement circuit check and, if any, the location of the abnormality.

The operation is allowed to proceed to the subsequent measurement process only when the remaining power of the battery 102 is higher than predetermined (e.g. 10% or higher) and the measurement circuits are normal. For example, when the remaining power of the battery 102 is insufficient, the plug of the AC-DC adaptor 3 should be connected to the outlet of the commercial AC power supply 5, or when there is an abnormality in the measurement circuits, the abnormality should be duly removed. After that, the operation is allowed to proceed to Step S4.

When the remaining power of the battery 102 is higher than predetermined and the measurement circuits are normal, if the examiner selects the function button A5 on the screen A with a mouse or other pointing device, or operates the keyboard to perform an operation having the same function (Step S4), then the operation enters the body composition measurement mode, and the initial screen A on the display unit 106 is replaced with a body composition measurement screen B (Step S5).

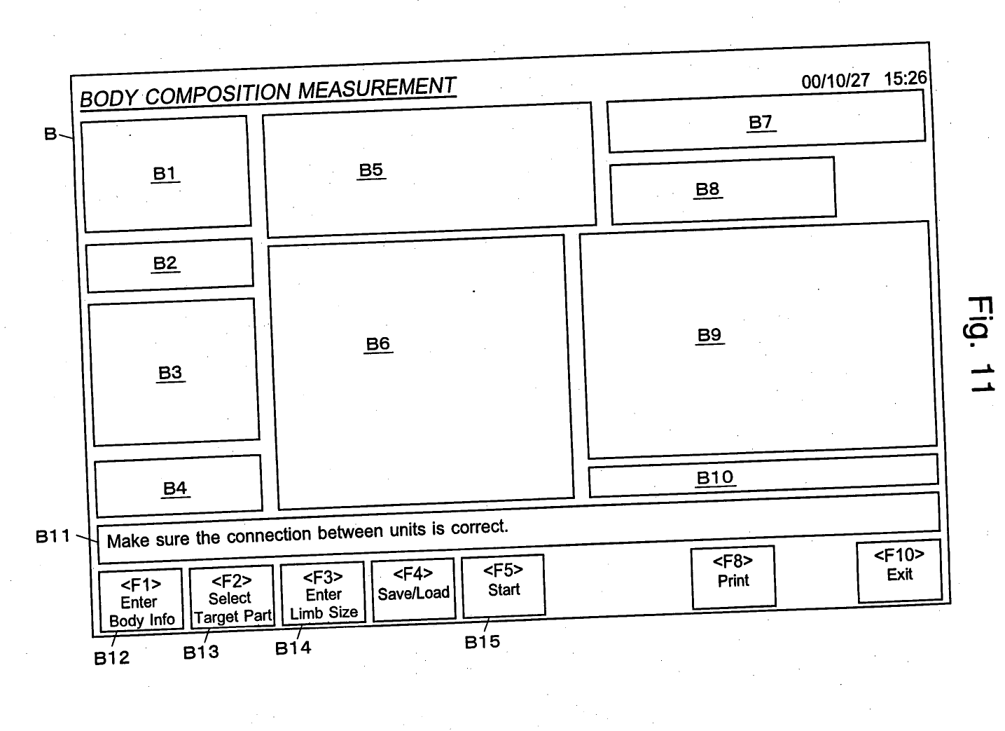

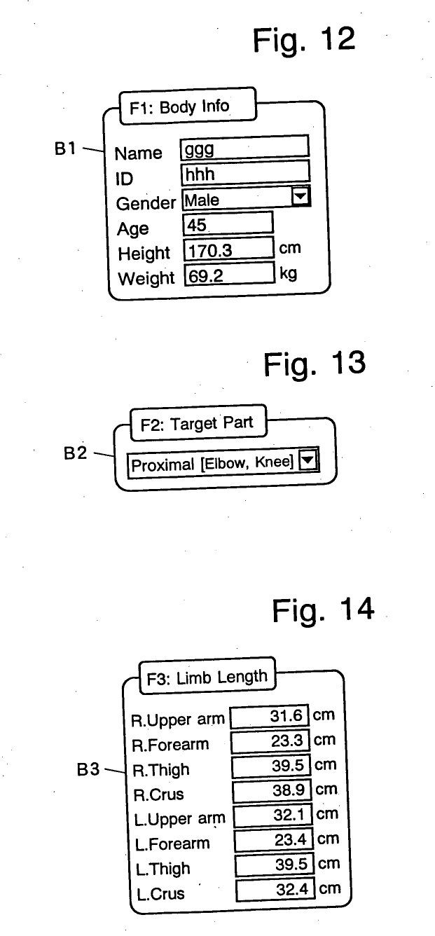

FIG. 11 is an outlined drawing of the body composition measurement screen B, and FIGS. 12-20 are detailed drawings of the main parts of the screen B.

In the measurement of the body composition, the subject is recommended to take a supine position on the bed or similar surface during the measurement. To eliminate the influence from the change in the body fluid balance, it is preferable to keep the subject resting in the above position for about five minutes. The four limbs should be fully extended and opened by the angle of about 30 degrees to keep the arms apart from the trunk and the legs apart from each other.

With the body composition measurement screen B displayed on the display unit 106, when the examiner selects the function button B12, a blinking cursor appears in the body information display area B1 having text boxes for entering and displaying the name and identifier (ID) of the subject and body specific information, such as gender, age, height and weight, to indicate what item should be entered. Looking at the screen, the examiner operates the keyboard to enter the name, ID and body specific information of the subject (Step S6).

When the height is entered, the lengths of the four limbs are calculated by predetermined formulae, the result of which is displayed in the text boxes of the limb length display area B3 shown in FIG. 14. When, for example, it is desired to enter the actually measured lengths of the limbs of the subject, the function button B14 should be selected. Then, a blinking cursor appears in a text box of the limb length display area B3, indicating what item should be entered. There, the value can be changed (Step S7). When the values are not changed, the values calculated as described above are used as the lengths of the limbs in the calculation, which will be described later.

Next, the examiner selects the “target part selection” function button B13 to select “distal”, “proximal” or “distal→proximal” in the text box of the target part display area B2 shown in FIG. 13. It is assumed here that the “distal→proximal” is selected in order to measure the aforementioned nine segments. It is of course possible to select “distal” or “proximal”.

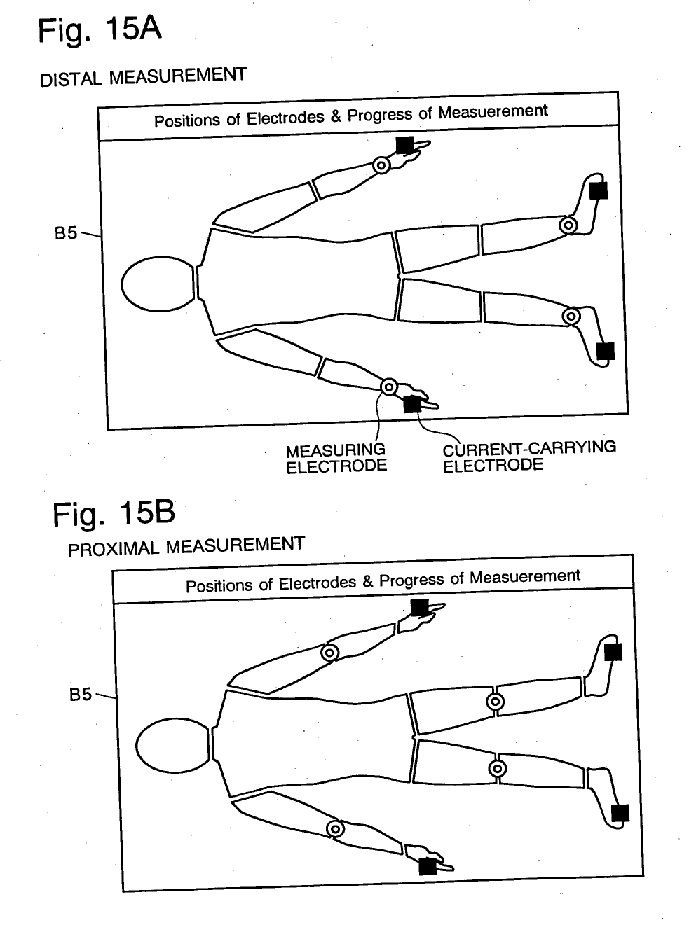

When all the body specific information has been entered, it is determined that the entry is completed (“Yes” in Step S9), and the electrode attachment positions for the distal measurement are indicated in the electrode attachment position display area B5 shown in FIG. 15 (Step S10). That is, the electrode attachment position display area B5 shows a figurative model of the human body divided into nine segments exclusive of the head and the fingertips of the hands and the feet, on which the positions to attach the electrodes are indicated by the two symbols: “▪” for the current-carrying electrode 10 and “⊚” for the measuring electrode 11. When the apparatus is standby for distal measurement, the symbols “⊚” indicative of the position for attaching the measuring electrodes are displayed at both wrists and both ankles, and the symbols “▪” indicative of the positions for attaching the current-carrying electrodes are displayed at the insteps of the feet, as shown in FIG. 15A. Referencing the displayed image, the examiner attaches the current-carrying electrodes 10 and the measuring electrodes 11 to the body of the subject.

After all the electrodes 10 and 11 have been attached to the body, the examiner operates the “start” function button B15 to give a command for starting the measurement (Step S11). In response to this operation, the measurement is automatically started. In advance of the measurement, the power line opening/closing relay 213 is opened (Step S12) and, slightly later than that, the signal opening/closing relays 201 are closed (Step S13). As a result, the commercial AC power supply 5 is disconnected from the main unit 2, and then the electrodes 10 and 11 are connected to the main unit 2. Therefore, if any trouble should happen, there is no possibility that the alternating current of 100V from the commercial AC power supply 5 enters the body of the subject. Also, during the subsequent measurement period, the entry of noises from the commercial AC power supply 5 can be prevented.

After that, the current-carrying electrodes 10 and the measuring electrodes 11 are switched by the current-carrying electrode selector 202 and the measuring electrode selector 204 so that the right arm, left arm, right leg, left leg and trunk are sequentially selected as the target part. A weak radio frequency current is supplied between two current-carrying electrodes 10 selected, and the electric potential generated by the current is measured one after another with two measuring electrodes 11. In the figurative model of the human body displayed in the electrode attachment position display area B5, all the segments selected as the target parts are shown as blinking gray images before the measurement. After that, segments for which the measurement has been completed are changed to lit green images. This makes it possible to know the progress of the measurement by simply looking at the displayed image.

In the measurement of the impedance of each target part, the measurement value is not stored into the memory until the impedance is stabilized to a certain extent. If the measurement value remains unstable for a long time and the measurement is not completed for any of the target parts even after a predetermined period of time, it is determined that the measurement is not performable (Step S15). When the measurement is completed for all of the five target parts, or when the measurement is completed for at least one target part after a predetermined period of time, the measurement is determined as completed (Step S17). In the case of determining that the measurement is not performable, it is probable that there is some abnormality in the measurement. Accordingly, an error message, such as “Measurement not performable” or “Operation abnormal”, is displayed in the message display area B112 (Step S16), and the measurement is terminated.

The process of Step S15 prevents the measurement from taking an abnormally long time because of an unstable measurement state. That is, at the moment the measurement has been continued for a certain period of time, if the measurement has been completed for some target parts, the values of the unmeasured parts are estimated from the measured data, and the measurement of the impedance itself is terminated. This operation prevents the subject from being overburdened.

After the measurement is completed, the signal line opening/closing relays 201 are opened (Step S18) to disconnect the electrodes 10 and 11 from the main unit 2. After that, the power line opening/closing relay 213 is closed (Step S19) to connect the AC-DC adaptor 3, which is connected to the commercial AC power supply 5, to the main unit 2. Therefore, the electrodes 10 and 11 are connected to the measurement circuits only during the measurement of the impedance, that is, only for a short period of time including a period of time for supplying the current through the body of the subject and measuring the voltage generated by the current. During the measurement of the impedance, the commercial AC power supply 5 is disconnected, and the main unit 2 and the PC 1 run on the DC power supplied from the battery 102.

After that, the impedances corresponding to the five target parts (right arm, left arm, right leg, left leg and trunk, in the case of distal measurement) and the body specific information are applied to the predetermined estimation formulae, or to the conversion tables corresponding to the formulae, to calculate the body composition, muscle masses of the limbs, ADL indices and body type (Step S20). This calculation may be performed using estimation formulae created using the body composition information obtained by the above-described MRI method. The estimation method is not always limited to this one. When only the distal measurement has been completed, it is impossible to perform a detailed estimation in which each arm is divided into upper arm and forearm and each leg is divided into thigh and crus. Accordingly, for these segments, values are roughly estimated from the body specific information and other information.

The numeral values obtained by the above estimation process are displayed in the measurement result display area B6, measurement value display area B7, ADL index display area B8, muscle mass display area B9 and body type display area B10 of the body composition measurement screen B, as described below (Step S21).

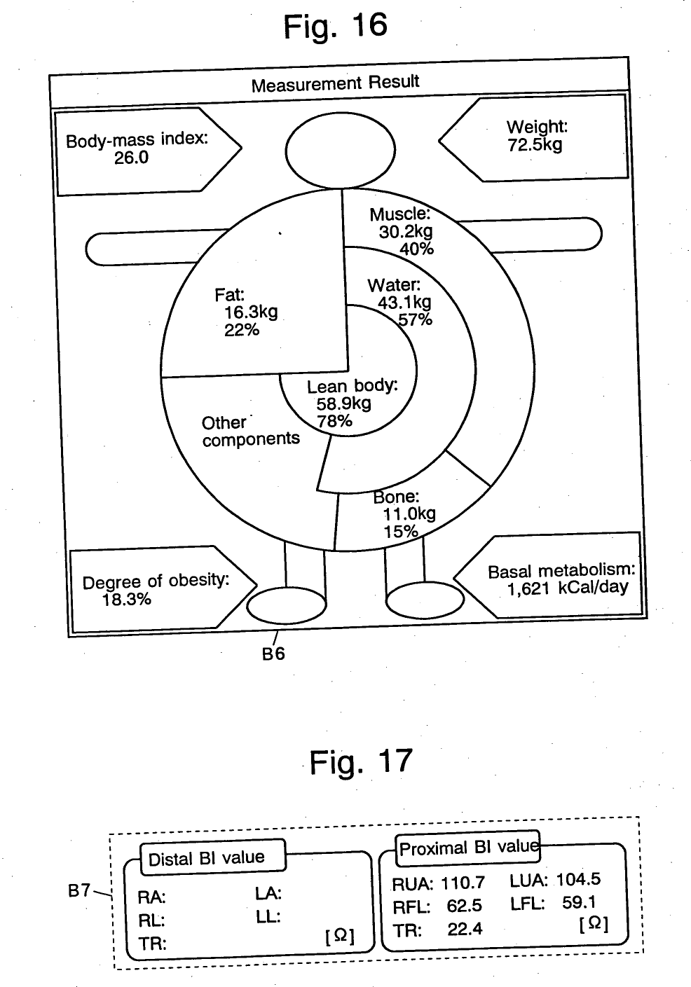

The impedance of each segment is diplayed in the left side of the measurement value display area B7 shown in FIG. 17. The information indicating the body composition of the entire body is displayed in the measurement result display area B6 shown in FIG. 16. In this area, a circle chart representing the human body in a deformational way is displayed to show the following three types of body composition ratios: fat, muscle, bone and other components; fat and lean body; and fat, water and other components. In addition, the following information is displayed: weight; body-mass index (BMI), derived from the height and other body specific information; degree of obesity; and estimated value of the basal metabolic rate.

The muscle mass display area B9 shown in FIG. 19 displays a bar chart showing the estimated muscle masses of the upper arms, forearms, arms, thighs, crura and legs on the right and left sides. In addition, the percentages of the right-and-left muscle masses are displayed to show the right-and-left balance. The ratio of the muscle mass of the arms to that of the legs is also displayed. These items of information make it easy to visually recognize the balance between the right and left muscles, from which it is possible to know, for example, which is the dominant arm or leg. The information can be used also for instant health examination. For example, when the right-and-left balance is abnormal, it is possible to guess that something is wrong with the health condition.

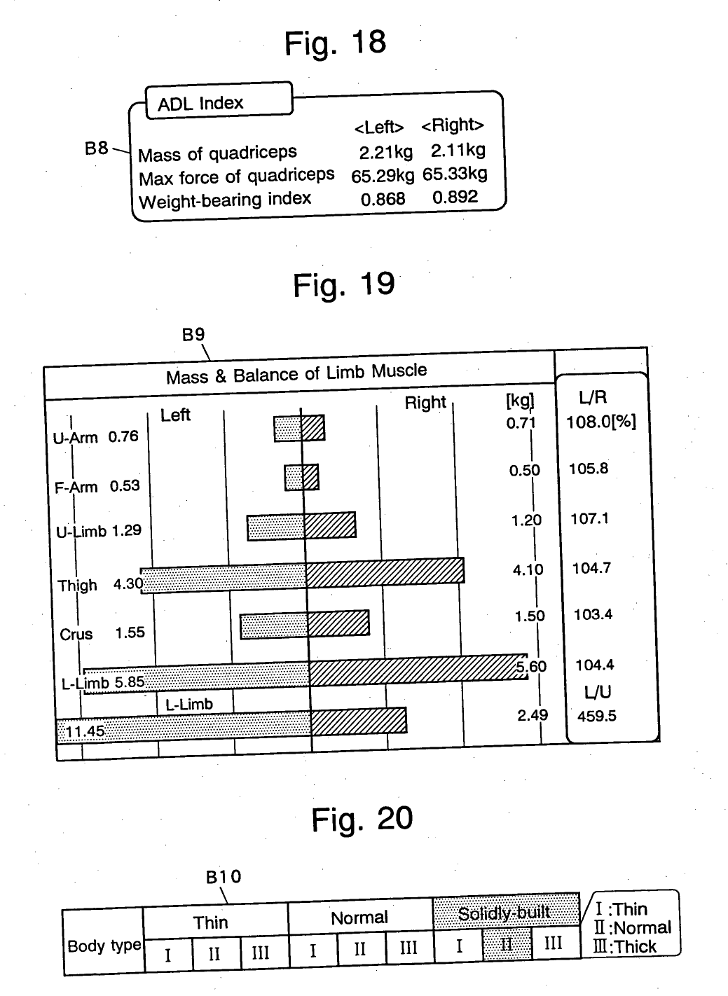

The ADL index display area B8 shown in FIG. 18 displays the masses and maximum forces of the right and left quadricepses, and the right and left weight bearing indices. These values serve as the ADL indices for evaluating the ability for the activity of daily living. The body type display area B10 shown in FIG. 20 displays the external body type as “thin”, “normal” or “solidly-built”, according to the body-mass index (BMI: W/H²) calculated from the weight (W) and height (H) entered as the body specific information. Furthermore, based on the body-fat ratio obtained as a result of the measurement, the state of deposits in the fat is displayed as “thin”, “normal” or “thick”. After the distal measurement is completed, some items of information that can be estimated at the moment can be displayed, even though both distal and proximal measurements are not completed.

When the distal measurement is completed, the attachment positions for the measuring electrodes 11 displayed on the figurative model of the human body in the electrode attachment position display area B5 are moved to the proximal positions shown in FIG. 15B (Step S22). That is, the symbols displayed at the right and left wrists and both ankles are moved to the right and left elbows and knees. Checking the change of the screen image, the examiner moves the four measuring electrodes 11 to the right and left elbows and ankles, and then operates the “start” function button B15 to give a command for starting the measurement (Step S23).

After that, the operation proceeds through Steps S24-S31, which correspond to Steps S12-S19 in the distal measurement, to perform the proximal measurement of the impedance of the limbs and the trunk. Now, the results of the distal and proximal measurements are completely available, so that the measurement values of the impedances corresponding to the nine segments can be obtained. Therefore, in the calculation of Step S32, the body composition and other information are estimated more accurately than when only the distal measurement has been completed. Then, the values thus calculated are displayed in place of the values shown in the measurement value display area B7, measurement result display area B6, ADL index display area B8, muscle mass display area B9 and body type display are B10 of the body composition measurement screen B (Step S33), and the measurement is terminated.

Thus, the present body composition measurement apparatus makes it possible to obtain accurate information reflecting the body composition and/or health condition in a relatively short period of time. Therefore, the subject experiences only a little physical or mental burden. Though the examiner must rearrange the electrodes in the course of the measurement, the work or operation is neither difficult nor complicated; the examiner has only to determine the attachment positions as instructed on the screen. Thus, the measurement can be easily performed. The information obtained by the measurement not only includes the body composition information, such as body fat mass or muscle mass, but also other information reflecting the health condition, such as the ADL index or the balance in muscle mass between the right and left side or upper and lower halves of the body. The information obtained thereby can be effectively used for various purposes, such as health management, physical training or rehabilitation.

It should be noted that a body impedance measurement apparatus according to the present invention may be constructed to include only a part of the body composition measurement apparatus described in the above embodiment and embody only a part of its functions. That is, the above embodiment is a mere example of the present invention, which can be variously altered or modified within the scope of the present invention, and the present invention obviously covers such alterations or modifications.

1. A body impedance measurement apparatus including a measuring unit for supplying a weak current through a body of a subject from current-carrying electrodes attached to the body and measuring a voltage generated by the current with measuring electrodes attached to the body, and a calculating unit for calculating an impedance of the body from a value of the current supplied through the body and a value of the voltage measured, comprising:
a) a power converter for converting AC power supplied from a commercial AC power supply to DC power;
b) a storage battery for storing the DC power and supplying the DC power as a driving power for the apparatus at least when the AC power is not supplied;
c) a power line opening/closing means for opening or closing a power line connecting the commercial AC power supply to the power converter or a power line connecting the power converter to the storage battery; and
d) a controller for opening the power line opening/closing means so that the driving power is supplied from the storage battery to each circuit of the apparatus at least during a period of time for measuring the voltage with the current supplied through the body.

2. The body impedance measurement apparatus according to claim 1, further including a signal line opening/closing means for opening or closing signal lines each connecting a measurement circuit included in the measuring unit with the current-carrying electrodes and the measuring electrodes, where the controller opens the signal line opening/closing means to disconnect the current-carrying electrodes and the measuring electrodes except for the period of time for measuring the voltage with the current supplied through the body.

3. The body impedance measurement apparatus according to claim 2, which is constructed to open the power line opening/closing means to disconnect the commercial AC power supply from the apparatus and then close the signal line opening/closing means to connect the current-carrying electrodes and the measuring electrodes to the measuring circuit unit before supplying the current through the body, and to open the signal line opening/closing means to disconnect the current-carrying electrodes and the measuring electrodes from the measuring circuit unit and then close the power line opening/closing means to connect the commercial AC power supply to the apparatus after the current supply is ended.

4. The body impedance measurement apparatus according to claim 1, wherein the power line opening/closing means and/or the signal line opening/closing means are electromagnetic relays.

5. The body impedance measurement apparatus according to claim 4, wherein the power line opening/closing means and/or the signal line opening/closing means are constructed electromagnetic relays that requires no driving current to open or close when the current is supplied through the body.

6. The body impedance measurement apparatus according to claim 1, wherein a predetermined control program is executed on a multi-purpose personal computer to implement a calculation process of the calculating unit, and the measuring unit is enclosed in a main unit having one and the same casing capable of communicating with the personal computer.

7. The body impedance measurement apparatus according to claim 6, wherein the storage battery is a built-in battery of the personal computer.

8. The body impedance measurement apparatus according to claim 6, comprising a serial interface as a communicating means between the personal computer and the main unit.

9. The body impedance measurement apparatus according to claim 8, wherein the communication means is an interface compliant with the USB standard, through which the main unit receives its driving power from the personal computer.

10. The body impedance measurement apparatus according to claim 6, further comprising a printing means for printing a measurement result or other information, wherein the personal computer communicates with the printing means by a wireless method.
